(19) United States
(12) Patent Application Publication
(10) Pub. No.: US 2005/0053579 A1
(43) Pub. Date: Mar. 10, 2005

(54) Novel synthetic chimeric fusion transgene with immuno-therapeutic uses

(76) Inventors: Jacques Galipeau, Town of mount-Royal (CA); John Stagg, Montreal (CA)

Correspondence Address: David S Resnick;Nixon Peabody, 100 Summer Street, Boston, MA, 02110, US

(21) Appl. No.: 10/493,344

(22) PCT Filed: Oct. 23, 2002
(86) PCT No.: PCT/CA02/01649

Related U.S. Application Data
(60) Provisional application No. 60/330,476, filed on Oct. 23, 2001.

(51) Int. Cl. A61K 38/19; A61K 38/20; C07K 14/54; C07K 14/52; C07K 14/525; C07K 14/53

(52) U.S. Cl. 424/85.1; 530/351; 424/85.2

(57) ABSTRACT

The present invention relates to an immuno-therapy conjugate which comprises A-c-B wherein: A and B are different and are compounds selected from the group consisting of cytokines, chemokines, interferons, their respective receptors or a functional fragment thereof; and c is a linker consisting of a bond or an amino acid sequence containing from 1 to 100 residues. The present invention also relates to a vaccine adjuvant comprising the immuno-therapy conjugate of the present invention. The present invention further relates to a method of reducing tumor growth, for inhibiting a viral infection and for improving immune response in a patient.

### BACKGROUND OF THE INVENTION

(a) Field of the Invention

The invention relates to a novel synthetic chimeric fusion gene and protein with immuno-therapeutic uses.

(b) Description of Prior Art

Research focusing on immunomodulation is attracting growing interest. DNA vaccines encoding for antigenic peptides have recently been developed as a novel vaccination technology against viral infections such as HIV (Ahlers JD. et al., Proceedings of the National Academy of Sciences of the United States of America. 94(20):10856-61, 1997 Sep. 30.), as well as against cancer (Strominger J L., Nature Medicine. 1(11):1140, 1995 November). For these next generation of vaccines based on poorly immunogeneic antigens, there is a great need for powerful adjuvants, both strong and safe, that can be used to enhance the immune response. Although many adjuvants such as LPS, LT and CT are used experimentally today (Vogel F R. Powell M F., [Review] Pharmaceutical Biotechnology. 6:141-228, 1995), most of them comprise a toxic fragment that is required for adjuvanticity, thus greatly hampering their clinical use.

The delivery of cytokine genes to enhance immune response to synthetic peptide vaccines may therefore represent an advantage over conventional adjuvants. Vaccination studies with genetically engineered cancer cells secreting cytokines such as IL-4, IL-6, IL-7, INF-γ, TNF-α, IL-12, GM-CSF or IL-2 (Dranoff G. et al., Proceedings of the National Academy of Sciences of the United States of America. 90(8):3539-43, 1993 April 15) (Irvine K R. et al., Journal of Immunology. 156(1):238-45, 1996 Jan. 1) have been shown to generate tumor-specific immune responses. Several studies have shown in addition that co-expressing some of these cytokines generated synergistic antitumor effects. Comparing the adjuvant effects of several cytokines on DNA vaccines revealed that the co-expression of GM-CSF and IL-2 genes induced the higher antibody titers and T cell proliferation response than other cytokine genes tested to date (Pan CH. et al., [Review] Journal of the Formosan Medical Association. 98(11):722-9, 1999 November). The co-expression of GM-CSF and IL-2 by tumor cells was also shown to induce potent synergistic antitumor effect (Lee S G. et al., Anticancer Research. 20(4):2681-6, 2000 Jul.-Aug.).

A bifunctional chimeric gene product borne from the fusion of GM-CSF and IL-2 cDNA may therefore display novel and potent immunostimulatory properties that could supersede that seen with either protein alone or expressed in combination. Granted, such a fusion sequence would be bereft of a true physiological role. However, the aim of cancer immunotherapy is to elicit as violent an immune reaction as possible against tumor. The idea of fusing GM-CSF with an interleukin is viable. As an example, the proprietary PIXY321 recombinant protein marketed by Immunex®) is a fusion of GM-CSF and IL-3 (Curtis B M. et al., Proceedings of the National Academy of Sciences of the United States of America. 88(13):5809-13, 1991 Jul. 1). This molecule was marketed as a stimulator of hematopoietic recovery from chemotherapy toxicity. Its successful bioengineering demonstrates the feasibility of fusing GM-CSF with interleukins.

GM-CSF was first described as a growth factor for granulocyte and macrophage progenitor cells. However, GM-CSF is also an important mediator for inflammatory reactions produced by T lymphocytes, macrophages and mast cells present at sites of inflammation (reviewed in Demetri G D. Griffin J D., [Review] Blood. 78(11):2791-808, 1991 Dec. 1). GM-CSF is a strong chemoattractant for neutrophils. It enhances microbicidal activity, phagocytotic activity and cytotoxicity of neutrophils and macrophages. An important feature of GM-CSF is that it greatly enhances the state of antigen presentation on dendritic cells, known to be crucial mediators of acquired immunity.

IL-2 on the other hand is an essential cytokine for the expansion of activated lymphocytes. IL-2 also supports the functional differentiation of mature lymphocytes, including CTL, NK cells and B cells. Moreover, IL-2 enhances CTL activity in activated primary CD8⁺ T cells through the fact that IL-2 upregulates mRNA for FasL, perforin and granzyme B, all of which are involved in the mechanism of CTL killing (Makrigiannis A P. Hoskin D W., Journal of Immunology. 159(10):4700-7, 1997 Nov. 15). NK cells also proliferate and upregulate their cytolytic activity in response to IL-2, but require relatively high doses of IL-2 since they do not express the high affinity receptor complex.

It would be highly desirable to be provided with a novel synthetic chimeric fusion transgene and protein with immuno-therapeutic uses.

### SUMMARY OF THE INVENTION

It is reported herein the successful engineering of a DNA plasmid encoding for a novel chimeric protein borne from the fusion of murine GM-CSF and murine IL-2 cDNA. The fusion was generated by restriction enzyme cloning, and resulted in a truncated murine GM-CSF cDNA at the 5′ end linked by a 3-bp linker to a the full length murine IL-2 cDNA at the 3′ end. Moreover, the expression of this fusion sequence in B16 murine melanoma cells led to the secretion of a GMCSF/IL2 fusion protein that greatly reduced the tumorigenicity of the cells in a syngeneic mouse model.

The novel immunostimulatory properties of this fusion transgene lead to an anti-cancer therapeutic effect. The present application shown that the nucleotide sequence encoding for GIFT can be utilized as a therapeutic transgene for gene therapy of cancer. The present application proposes that the fusion transgene nucleotide sequence can be utilized for: (i) genesis of cell and gene therapy biopharmaceuticals for treatment of cancer, (ii) as a genetic immunoadjuvant to DNA vaccine technologies for use in the prevention and treatment of cancer or infectious diseases in humans and other mammals and, (iii) as a genetic immunoadjuvant for production of commercially valuable monoclonal and polyclonal antibodies in mammals.

In accordance with the present invention there is provided an immuno-therapy conjugate which comprises:
A-c-B
wherein:
A and B are different and are compounds selected from the group consisting of cytokines, chemokines, interferons, their respective receptors or a functional fragment thereof; and
c is a linker consisting of a bond or an amino acid sequence containing from 1 to 100 residues.

The conjugate in accordance with a preferred embodiment of the present invention, wherein the cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

The conjugate in accordance with a preferred embodiment of the present invention, wherein the chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

The conjugate in accordance with a preferred embodiment of the present invention, wherein the interferon is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

In accordance with the present invention, there is provided an immuno-therapy fusion cDNA encoding the immunotherapy conjugate of the present invention.

In accordance with the present invention, there is provided a vaccine adjuvant for DNA vaccination which comprises the conjugate of the present invention.

The vaccine adjuvant in accordance with a preferred embodiment of the present invention, wherein the vaccination is against an infectious organism.

The vaccine adjuvant in accordance with a preferred embodiment of the present invention, wherein the infectious organism is selected from the group consisting of: viruses, bacteries, mycobacteria, protozoa and prions.

The vaccine adjuvant in accordance with a preferred embodiment of the present invention, wherein the virus is selected from the group of Influenza virus, Hepatitis A virus, Hepatitis B virus, Hepatitis C virus, HIV, Yellow fever virus, Aphthovirus and Filovirus.

The vaccine adjuvant in accordance with a preferred embodiment of the present invention, wherein the vaccination is against malignancies, wherein the malignancies having at least one immunogen associated thereto.

In accordance with the present invention, there is provided a vaccine adjuvant for vaccination, which comprises the fusion cDNA of the present invention.

In accordance with the present invention, there is provided a method for reducing tumor growth in a patient, the method comprising administering to the patient a therapeutically effective amount of the conjugate of the present invention.

In accordance with the present invention, there is provided a method for reducing tumor growth in a patient, the method comprising administering to the patient a therapeutically effective amount of normal autologous patient-derived cells engineered ex vivo to integrate and express the fusion cDNA of the present invention.

In accordance with the present invention, there is provided a method for inhibiting a viral infection in a patient, the method comprising administering to the patient a therapeutically effective amount of the conjugate of the present invention.

In accordance with the present invention, there is provided a method to inhibit a viral infection in a patient, the method comprising administering to the patient a therapeutically effective amount of the fusion cDNA of the present invention using a gene delivery technique.

The method in accordance with a preferred embodiment of the present invention, wherein the gene delivery technique is selected from the group consisting of: recombinant viral based vectors and plasmid DNA delivery methods.

In accordance with the present invention, there is provided a method to inhibit a viral infection in a patient, the method comprising administering to the patient a therapeutically effective amount of normal autologous patient-derived cells engineered ex vivo to integrate and express the fusion cDNA of the present invention.

In accordance with the present invention, there is provided a method to allow production of antigen-specific antibodies, the method comprising the administration of the species-specific fusion cDNA of claim 5 with the cDNA of the antigen or functional fragment thereof in experimental mammals.

In accordance with the present invention, there is provided a method to inhibit a viral infection in a patient, the method comprising administering to the patient a therapeutically effective amount of the fusion cDNA of the present invention using a gene delivery technique.

In accordance with the present invention, there is provided the use of a therapeutically effective amount of the conjugate of the present invention for reducing tumor growth in a patient.

In accordance with the present invention, there is provided the use of a therapeutically effective amount of the fusion cDNA of the present invention with a gene delivery technique for reducing tumor growth in a patient.

In accordance with the present invention, there is provided the se of a therapeutically effective amount of normal autologous patient-derived cells engineered ex vivo to integrate and express the fusion cDNA of the present invention for reducing tumor growth in a patient.

In accordance with the present invention, there is provided the use of a therapeutically effective amount of the conjugate of the present invention for inhibiting a viral infection in a patient.

In accordance with the present invention, there is provided the use of a therapeutically effective amount of the fusion cDNA of the present invention with a gene delivery technique to inhibit a viral infection in a patient.

In accordance with the present invention, there is provided the use of a therapeutically effective amount of normal autologous patient-derived cells engineered ex vivo to integrate and express the fusion cDNA of the present invention to inhibit a viral infection in a patient.

In accordance with the present invention, there is provided the use of species-specific fusion cDNA of the present invention with the cDNA of antigen or functional fragment thereof to allow production of antigen-specific antibodies in mammals.

In accordance with the present invention, there is provided the use of a therapeutically effective amount of the conjugate of the present invention to improve immune response in a patient.

For the purpose of the present invention the following terms are defined below.

The term “subject” is intended to mean humans, mammals and/or vertebrates.

The term “functional fragment” is intended to mean a fragment that as conserved the same activity as the entire product.

### BRIEF DESCRIPTION OF THE DRAWINGS

FIG. 1 illustrates pGMCSF and pIL2 restriction enzyme maps;

FIG. 2 illustrates pGMCSF EcoRI digest on agarose gel;

FIG. 3 illustrates pGMCSF EcoRV digest on agarose gel, after EcoRI digestion;

FIG. 4 illustrates pIL2 PstI digest;

FIG. 5 illustrates pIL2 EcoRI digest (after Pst1 and S1 nuclease);

FIG. 6 illustrates the ligation of mGM-CSF to mIL-2;

FIG. 7 illustrates the ligation product Hindel digest;

FIG. 8 illustrates pJS330 confirmation digest;

FIG. 9 illustrates pJS330 restriction map;

FIG. 10 illustrates the amino acid sequence of a schematic fusion protein showing the positive sequencing of the fusion between mouse GM-CSF cDNA and mouse IL-2 cDNA;

FIG. 11 illustrates pJS330 XhoI-Hpal digest and AP2 BamHI digest;

FIG. 12 illustrates pJS4 confirmation digest;

FIG. 13 illustrates pJS4 restriction map;

FIG. 14 illustrates the secretion of the fusion protein by the JS4-transduced B16 cells;

FIG. 15 illustrates immunoblotting of the fusion protein with monoclonal antibodies against mouse IL-2 or mouse GM-CSF;

FIG. 16 illustrates the antitumor effect of the mGM-CSF/mIL2 fusion sequence when expressed in B16 melanoma cells;

FIG. 17 illustrates H&E staining of 5 μm tumor sections from mice injected s.c. with 10⁶ B16 cells engineered to secrete the mGMGSF/mIL2 fusion protein and GFP (FIGS. 17B and 17D) or engineered to secrete GFP only (FIGS. 17A and 17C); and

FIG. 18 illustrates the level of secretion of the fusion protein determined in vitro by ELISA.

### DETAILED DESCRIPTION OF THE INVENTION

In accordance with the present invention, there is provided a novel synthetic chimeric fusion transgene with immuno-therapeutic uses. It is therefore proposed that a bifunctional chimeric gene product borne from the fusion of GM-CSF and IL-2 cDNA may display novel and potent immunostimulatory properties that could supersede that seen with either protein alone or expressed in combination. Further, a fusion transgene will guarantee equimolar production of GM-CSF and IL-2 by all engineered cells. This is of significance, since independent transfer of IL-2 and GM-CSF is random in distribution, and it is only by chance that any gene-transfected cell express both protein.

Materials and Methods

Mouse IL2 and mouse GM-CSF cDNAs were purchased from the National Gene Vector Laboratories (NGVL, The University of Michigan). The synthesis of the fusion protein expression plasmid, namely pJS330, was as follow.

Cloning pIL2

The 557-bp IL2 cDNA was excised by Pst1-Swa1 restriction digest and ligated to the 3970-bp pEGFP-N1 (Clontech, Palo Alto, Calif.) fragment generated with Not1, Klenow fill-in and Pst1. This murine IL2 expression plasmid is referred to as pIL2 in the following text.

Cloning pGMCSF

The 462-bp GM-CSF cDNA was excised by Sal1-BamH1 restriction digest and ligated into the previously reported plasmid AP2 after XhoI-BamH1 digest. Briefly, AP2 is a plasmid encoding for a bicistronic murine retrovector that incorporates a multiple cloning site, allowing insertion of a cDNA of interest. This murine GM-CSF expression plasmid is referred to as pGMCSF in the following text.

Cloning pJS330

A 398-bp fragment from pGMCSF containing the cDNA for the mouse GM-CSF (truncated 33-bp prior to the stop codon) was excised by EcoRI followed by EcoRV. This truncated cDNA was ligated to the 5′ end of the mIL2 gene into pIL2. Prior to ligation, pIL2 was digested with Pst1 (cutting 3-bp prior to IL2 start codon), followed by S1 nuclease to remove single stranded DNA, and EcoRI digest. 30 μl of the 398-bp of pGMCSF was added to 551 of the 4518-bp of pIL2 in the presence of DNA ligase for 16 hours at 14° C. Transformation of the ligation product was carried on in DH5α competent bacteria, and the bacteria subsequently were plated on agar. Colonies were grown for 12 hours and individual clones were picked and grew in LB broth for 12 hours. The DNA was then isolated using a commercial kit. The ligation product is referred to as pJS330 in the following text and encodes the fusion protein mGM-CSF/mIL2.

The fusion mGM-CSF/mIL2 DNA coding sequence within pJS330 was subsequently sent for sequencing at the Guelph Molecular Supercentre (University of Guelph, Ontario). The two sequencing primers used (i.e. 5′-ACAGCCAGCTACTACCAGAC-3′ [P1] (SEQ ID NO:1) and 5′-CGCTACCGGACTCAGATCTC-3′ [P2] (SEQ ID NO:2)) were generated at the Sheldon Biotechnology Center (McGill University, Montreal).

Cloning pJS4

A 1090-bp fragment from pJS330 containing the fusion protein coding sequence was excised by XhoI-HpaI restriction digest and ligated into AP2 after BamH1, Klenow fill-in and XhoI. The ligation product is a retrovector plasmid referred to as pJS4 that allows for the expression of mGM-CSF/mIL2 fusion protein and GFP, as well as the generation of retrovectors when transfected into packaging cell lines.

Fusion Protein Expression

The expression and secretion of the mGM-CSF/mIL2 fusion protein was confirmed by ELISA. 5 μg of the retrovector plasmid pJS4 or AP2 were digested with PstI and co-transfected with 0.5 μg of pJ6ΩBleo plasmid into GP+E86 retrovector packaging cells (American Type Culture Collection [ATCC]) with the use of Lipofectamine™ (Life Technologies, Inc.). Transfected cells were subsequently selected in DMEM media (10% heat-inactivated FBS plus 50 units/ml of Pen-Strep™) supplemented with 100 μg/ml Zeocin™ (Invitrogen, San Diego, Calif.) for 4 weeks. Resulting stable producers generated ecotropic retroviral titers of 10⁵ cfu/ml. GP+AM12 retrovector packaging cells (ATCC) were transduced with 10 ml of fresh supernatant from pJS4 or AP2-transfected GP+E86 (plus 6 μg/ml Lipofectamine) twice daily for 3 consecutive days. Resulting stable producers generated amphotropic viral titers of 10⁵ cfu/ml. B16 murine melanoma cells were transduced with 10 ml of fresh supernatant from pJS4 or AP2-transduced GP+AM12 (plus 6 μg/ml Lipofectamine) twice daily for 6 consecutive days. One week later, 24 hours old supernatant was collected from B16-transduced cells, namely B16-JS4 and B16-AP2, and the cells counted by hemacytometer. The collected supernatant was frozen until thawed for ELISA detecting the presence of mGM-CSF protein (Biosource, San Diego, Calif.) or mIL-2 protein (Biosource, San Diego, Calif.) in the supernatant according to the manufacturer's instructions.

B16 Modified Cells In Vivo Implantation

Murine B16 engineered melanoma cells secreting the fusion protein and the reporter GFP (B16-JS4 cells) were injected subcutaneously (s.c.) in syngenic immunocompetent C57bl/6 mice. As a control, B16 melanoma cells expressing GFP only (B16-AP2 cells) were injected. Prior to implantation, the cells were trypsinized and centrifuged at 2000 rpm for 5 minutes in the presence of 10% FBS DMEM media. The cells were then resuspended in PBS. One million cells (in 100 μl PBS) were injected per mouse using a 25^{5/8} gauge syringe. Seven mice per group were injected subcutaneously and tumor volume was measured over time with a vernier caliper using the following formula: tumor volume=tumor length×(tumor width)²/2.

Histology

Control tumors were resected at day 20 post-implantation while tumors expressing the fusion protein were resected at day 52 post-implantation. Resected tumors were immediately fixed in 10% formalin, and subsequently embedded in paraffin, cut in 5 μm-thick sections and stained with hematoxylin and eosin (H&E). Four sections per tumor were blindly examined microscopically by a pathologist to characterize the immune infiltration.

Results

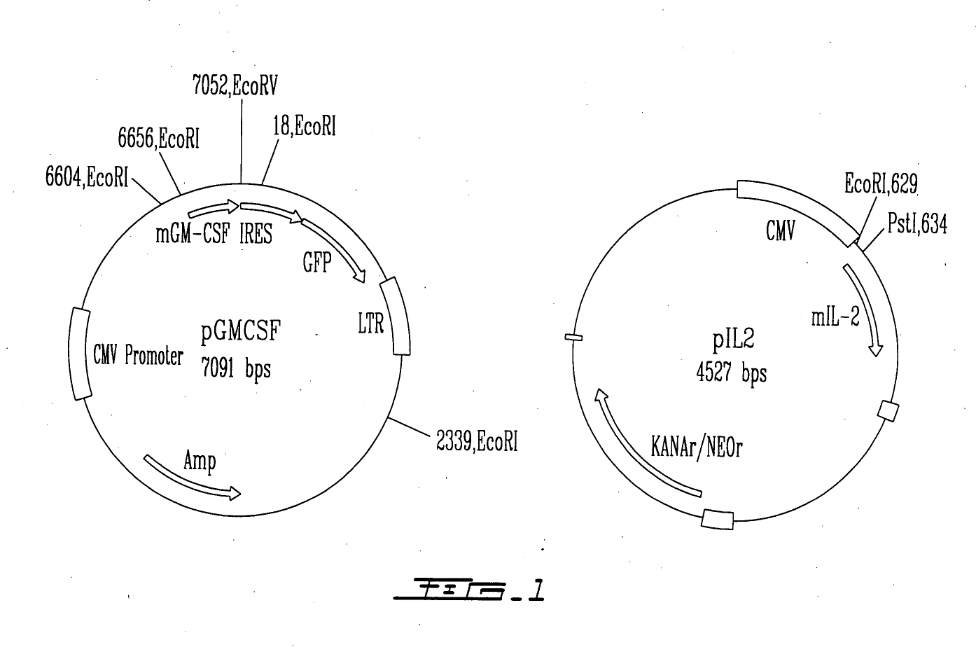

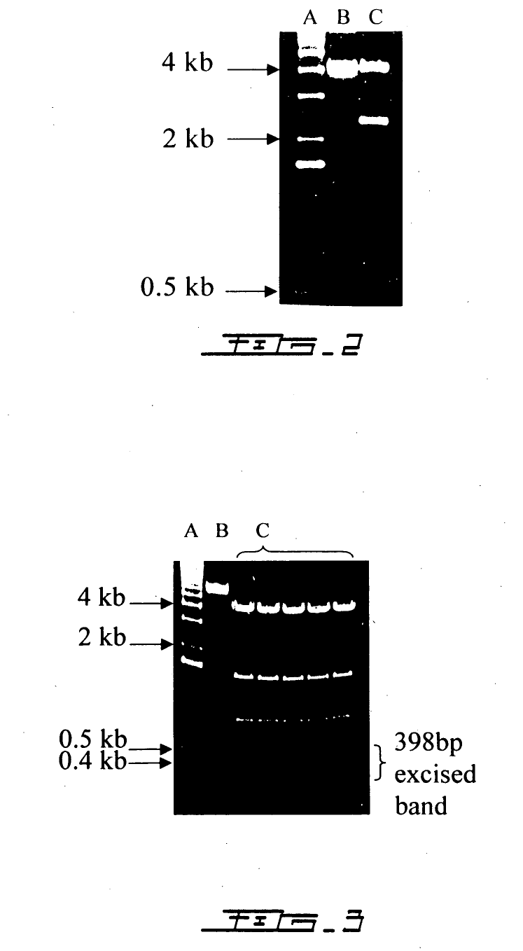

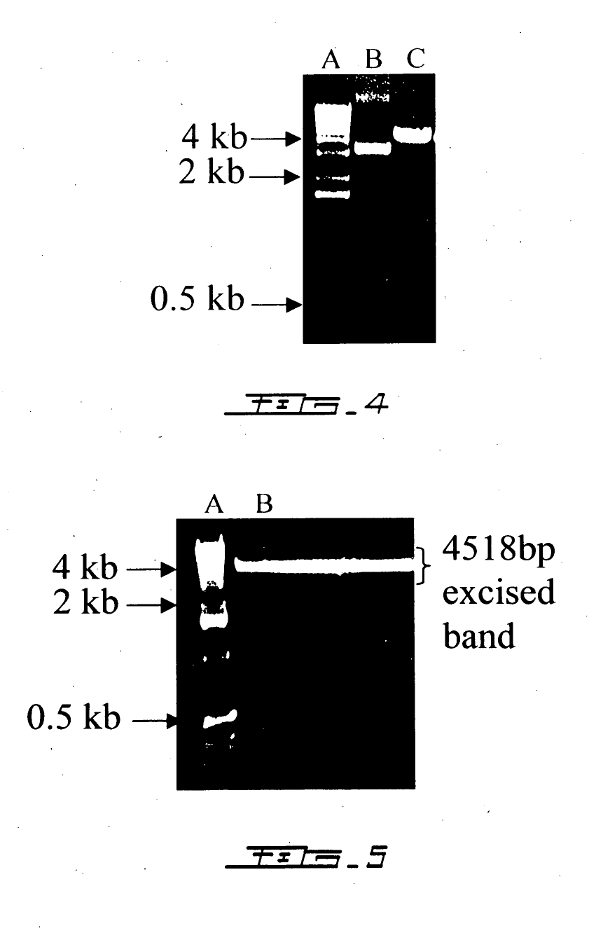

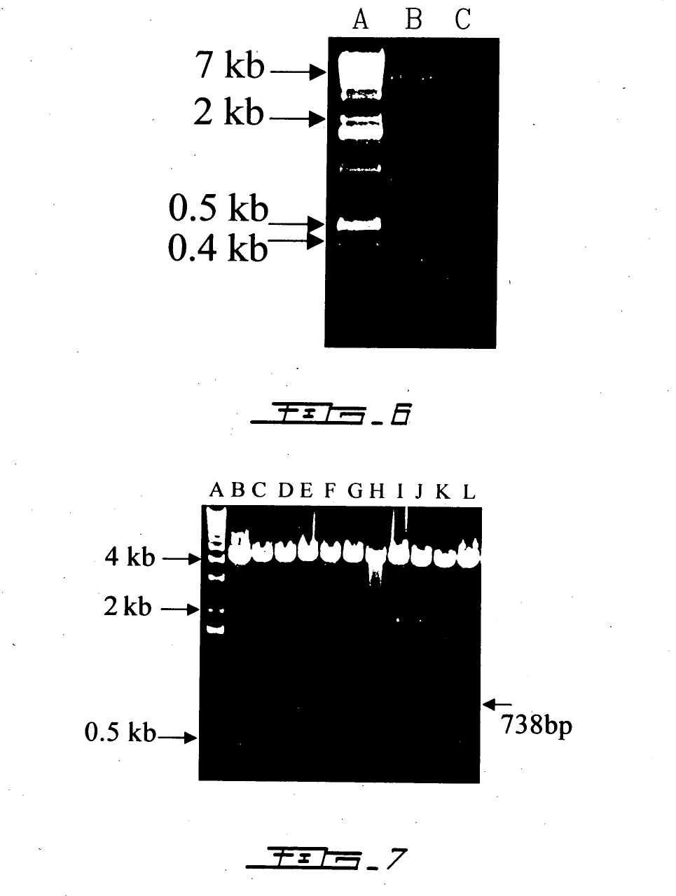

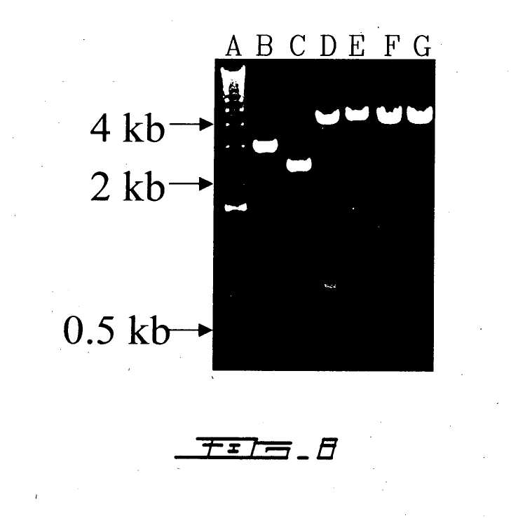

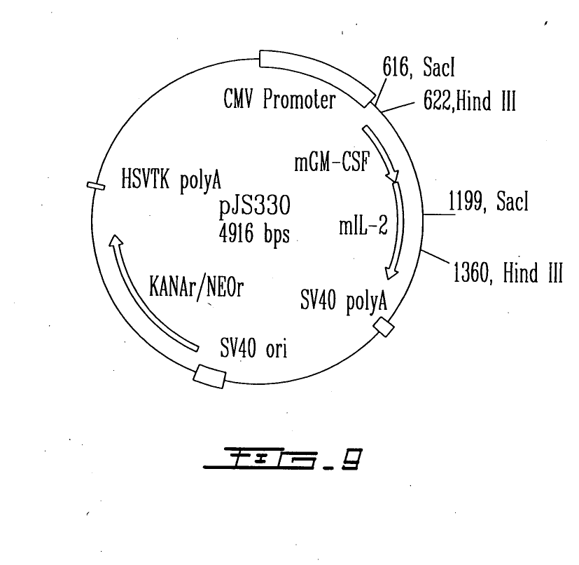

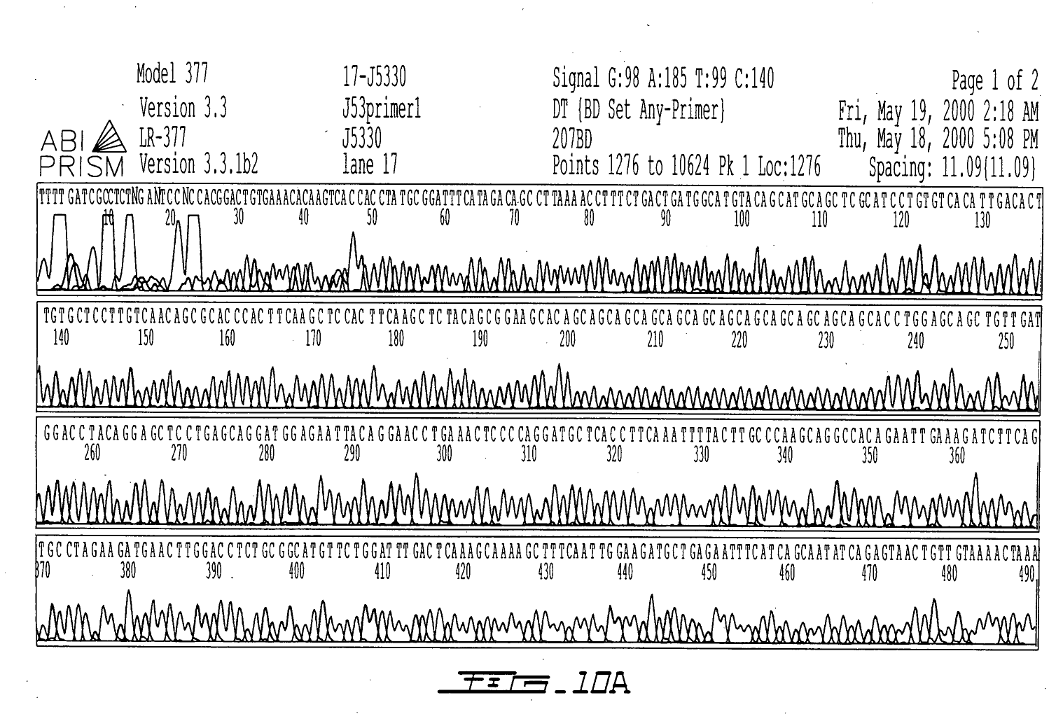

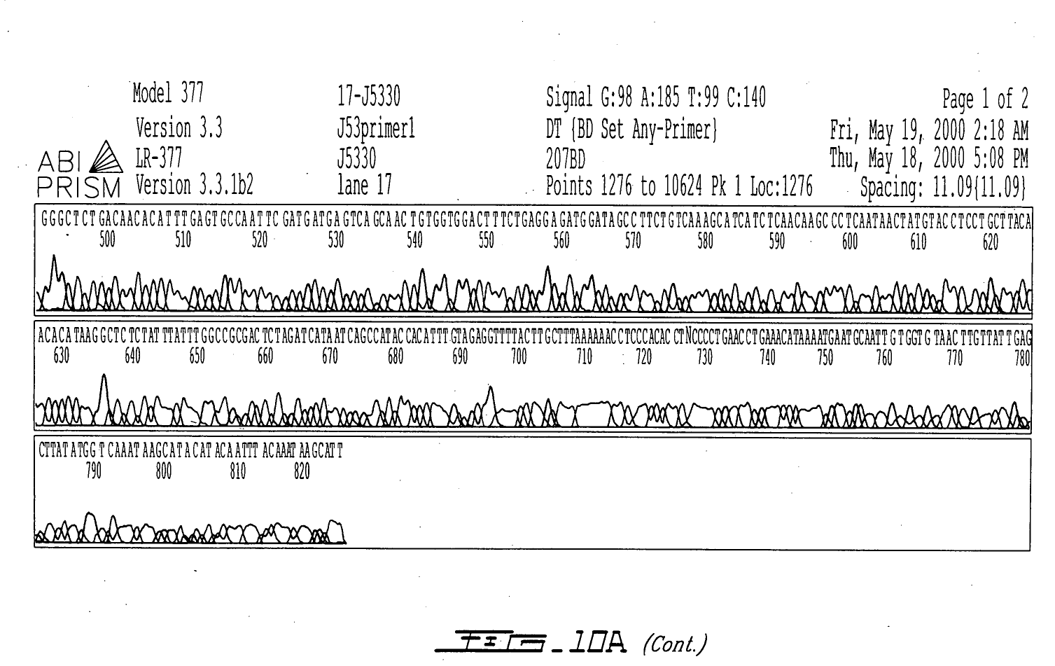

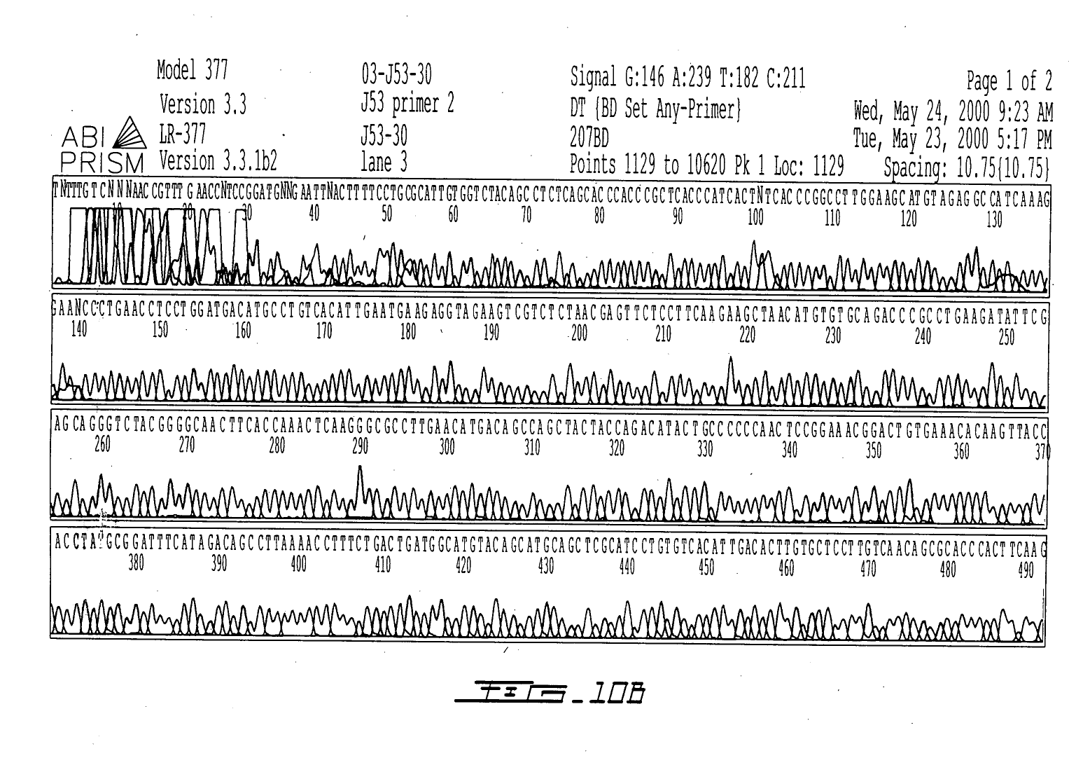

The cDNA for mouse GM-CSF and mouse IL2 were purchased from the National Gene Vector Laboratories and subsequently subcloned in two distinct expression plasmids, namely pGMCSF and pIL2 (FIG. 1). pGMCSF expression plasmid was first digested with EcoRI restriction enzyme and a sample run on agarose gel for confirmation (FIG. 2). In FIG. 2, column A is 1 kb DNA ladder, column B is uncut pGMCSF, column C is 52 bp, 453 bp, 2321 bp and 4265 bp fragments of pGMCSF EcoRI (Eth.Br. agarose gel 0.8%). The remaining DNA was then digested with EcoRV (FIG. 3) and the 398-bp band containing the mGM-CSF sequence was excised and purified. In FIG. 3, column A is 1 kb DNA ladder, column B is uncut pGMCSF, column C is 398 bp, 878 bp, 1443 bp and 4265 bp fragments of pGMCSF. Meanwhile, the pIL2 expression plasmid was linearized with PstI and a sample was run on agarose gel for confirmation (FIG. 4). In FIG. 4, column A is 1 kb DNA ladder, column B is uncut pIL2 and column C is linear pIL2 after PstI. The remaining DNA was then deprived from any single-chain overhangs using S1 nuclease. Subsequently, the DNA was digested with EcoRI and the 4518-bp band containing the mIL2 cDNA sequence was excised and purified (FIG. 5). In FIG. 5, column A is 1 kb DNA and column B is pIL2 4518 bp Band (Eth. Br. agarose gel 0.8%). 5 μl of the 398-bp DNA and 5 μl of the 4518-bp DNA were run in parallel on agarose gel prior to ligation (FIG. 6). In FIG. 6, column A is 1 kb DNA ladder, column B is 4518 bp band of pIL2 and column C is 398 bp band of pGMCSF (Eth.Br. agarose gel 0.8%). Following transformation of the ligation product in competent AH5α bacteria, 40 individual clones were screened for the presence of the fusion sequence plasmid. The collected DNA was digested with Hind III for a first screen of a potential clone encoding the correct fusion sequence (FIG. 7). In FIG. 7, column A is 1 kb DNA ladder, columns B to L are clones 21 to 31 respectively. Expected bands for pJS330 are 738 bp and 4178 bp (Eth.Br. agarose gel 0.8%). Clone number 30 was identified as positive, and further used for confirmation with SacI (FIG. 8). In FIG. 8, column A is 1 kb DNA ladder, column B is pJS330 uncut, column C is pIL2 uncut, column D is pJS330 HindIII digest (expected bands 583 bp and 4333 bp), column F is pIL2 HindIII and G is pIL2 Sacl. (Eth.Br. agarose gel 0.8%). FIG. 9 is a restriction enzyme map of the plasmid pJS330 showing the sites used for confirmation.

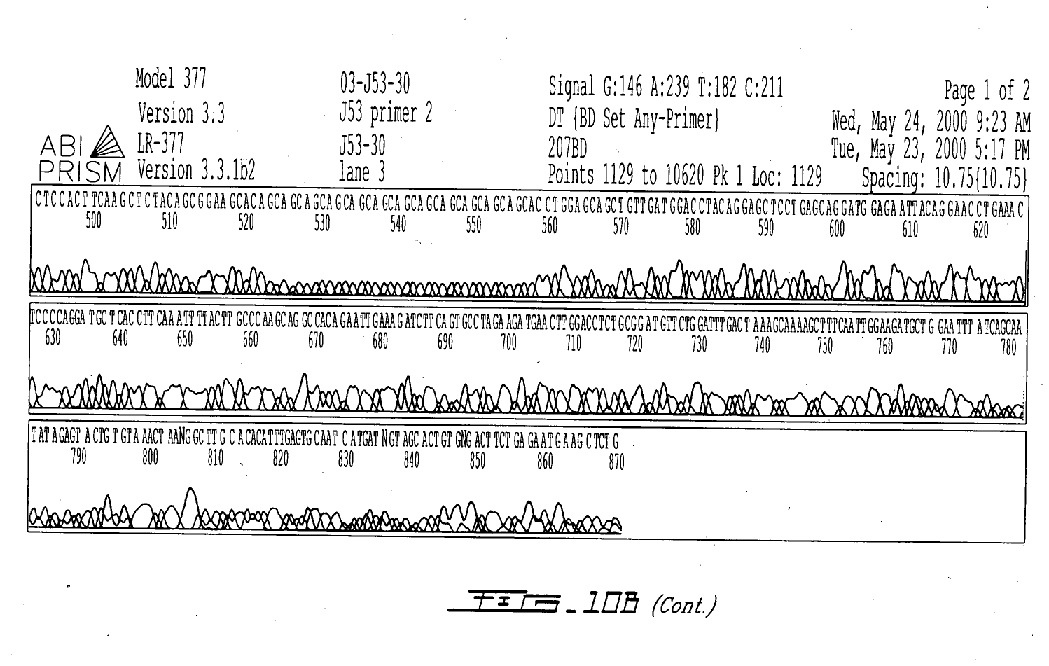

The DNA of clone number 30, namely pJS330, that showed to be positive by restriction enzymes for the presence of the fusion gene, was sent for sequencing using two distinct primers. Sequencing primer 1 (P1) is complementary to a 20-bp sequence 5′ of the expected glycine linker between mGM-CSF and mIL2. Sequencing primer 2 (P2) is complementary to a 20-bp sequence 5′ of the start codon of mGM-CSF. FIG. 10 represents the complete sequence analysis of the novel synthetic fusion transgene. In FIG. 10, A is the sequence analysis obtained from P1, B is the sequence analysis obtained from P2, and C is a schematic illustration of the predicted amino acid sequence.

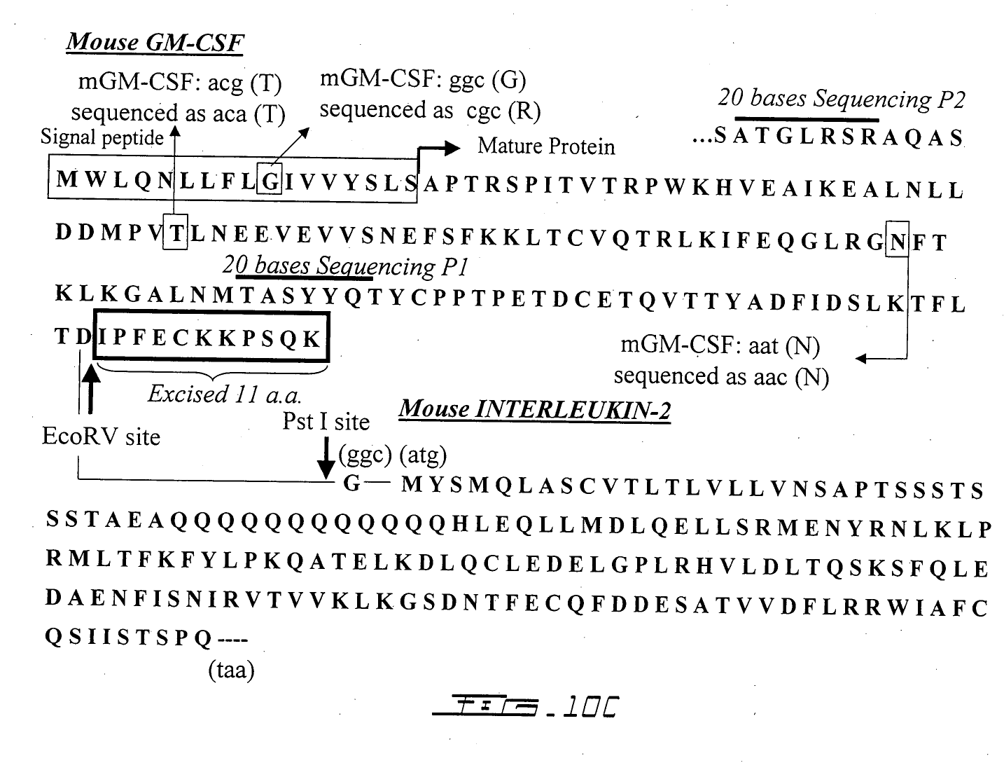

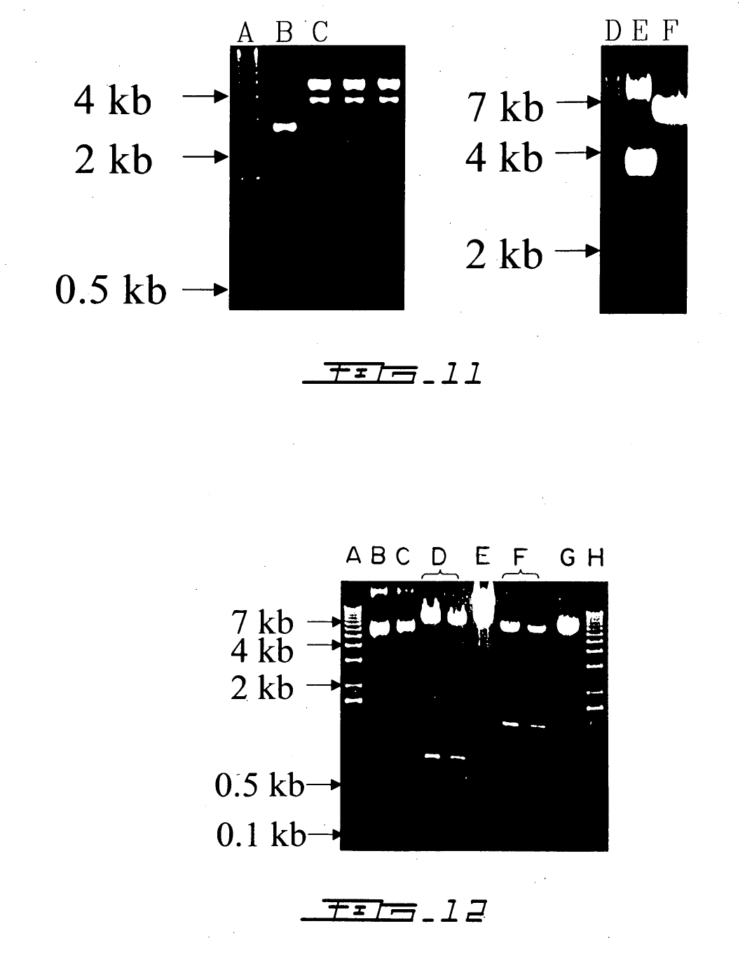

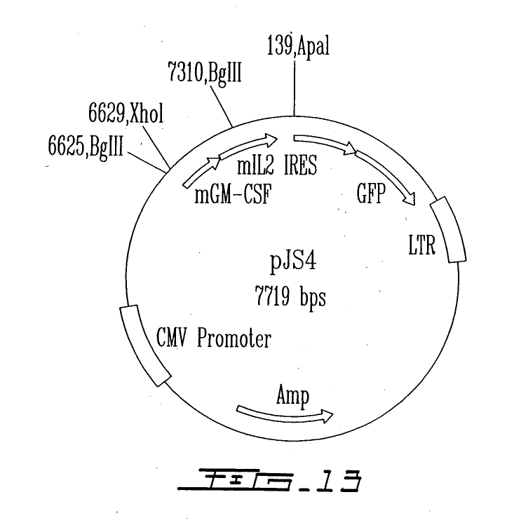

In order to engineer cancer cells to express this fusion gene, it has been generated a retrovector plasmid that encodes the mGMCSF/mIL2 fusion and the reporter GFP. The plasmid pJS330 was digested with XhoI-Hpal and the 1090-bp band containing the fusion gene was excised and purified (FIG. 11). In FIG. 11, column A is 1 kb DNA ladder, column B is pJS330 uncut, column C is 1090 bp and 3826 bp fragments of pJS330 XhoI-HpaI, column D is 1 kb DNA ladder, column E is AP2 uncut and column F is AP2 BamHI (Eth. Br. Agarose gel 0.8%). AP2 was first linearized with BamH1, then single-chained overhangs were filled-in, and the DNA digested with EcoRI. The two fragments (from pJS330 and AP2) were ligated, and the ligation product (pJS4) screened with BgIII and XhoI-ApaI digests (FIG. 12). In FIG. 12, column A is 1 kb DNA ladder, column B is pJS4 uncut, column C is AP2 uncut, column D is pJS4 BgIII digest (expected bands 685 bp and 7034 bp), column E is AP2 BbIII digest, column F is pJS4 XhoI-ApaI digest (expected bands 1233 bp and 6486 bp), column G is AP2 XhoI-ApaI digest and column H is 1 kb DNA ladder (Eth. Br. agarose gel 0.8%). FIG. 13 is a restriction enzyme map of the plasmid pJS4 showing the sites used for confirmation.

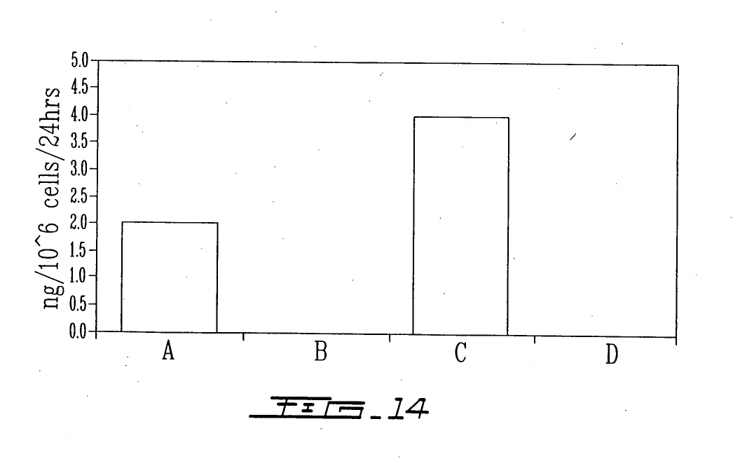

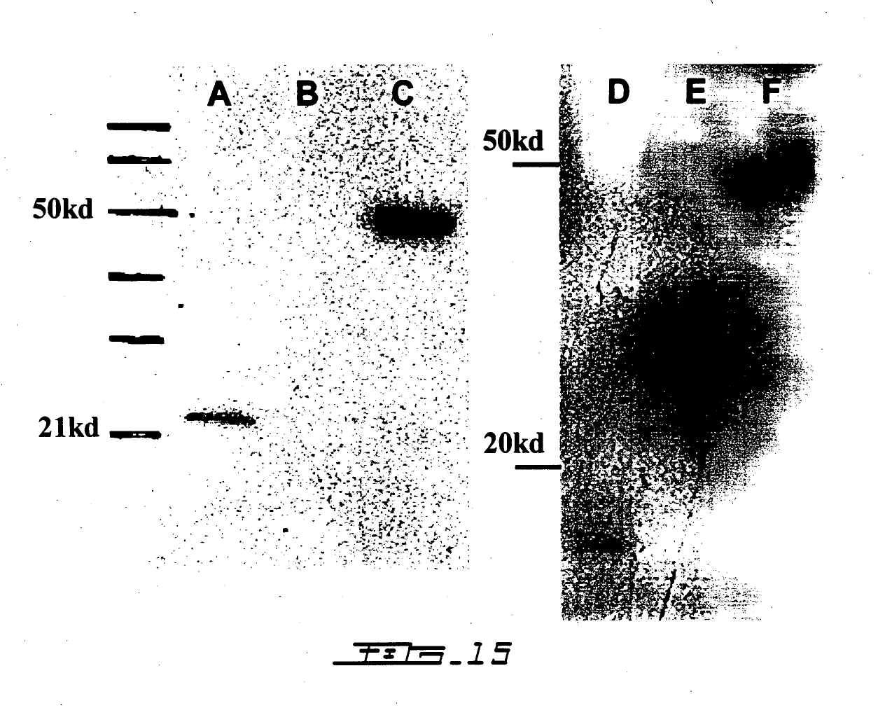

The retrovector plasmid pJS4 encoding the fusion sequence was transfected into GP+E86 packaging cells and the supernatant used to transduced GP+AM12 packaging cells. The supernatant of GP+AM12 was used to transduce B16 murine melanoma cells. The JS4-transduced B16 cells were assessed for secretion of the fusion protein by ELISA. The supernatant from B16-JS4 cells was positive for GM-CSF and IL-2 by ELISA confirming the secretion of the fusion protein (FIG. 14). In FIG. 14, A is the concentration of IL-2 produced by B16-JS4 cells, B is the concentration of IL-2 produced by non-modified B16 cells, C is the concentration of GM-CSF produced by B16-JS4 cells and D is the concentration of GM-CSF produced by naïve B16 cells. The molecular weight of the fusion protein was determined to be between 43 and 48 kilo Dalton (kD) by immunoblotting with monoclonal antibodies against mouse IL-2 or mouse GM-CSF (FIG. 15). In FIG. 15, A is recombinant mouse IL-2 probed against IL-2, B is recombinant mouse GM-CSF probed against IL-2, C is the fusion protein from B16-JS4 supernatant probed against IL-2, D is recombinant mouse GM-CSF probed against GM-CSF, E is recombinant mouse IL-2 probed against GM-CSF and F is the fusion protein from B16-JS4 supernatant probed against GM-CSF.

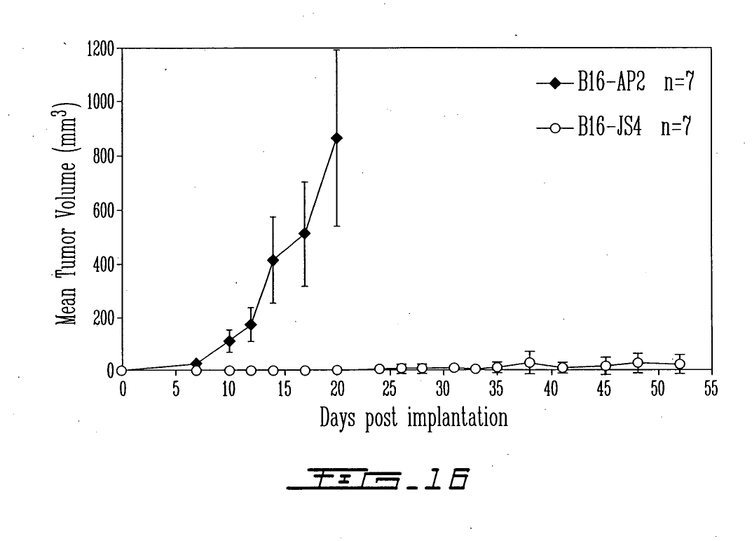

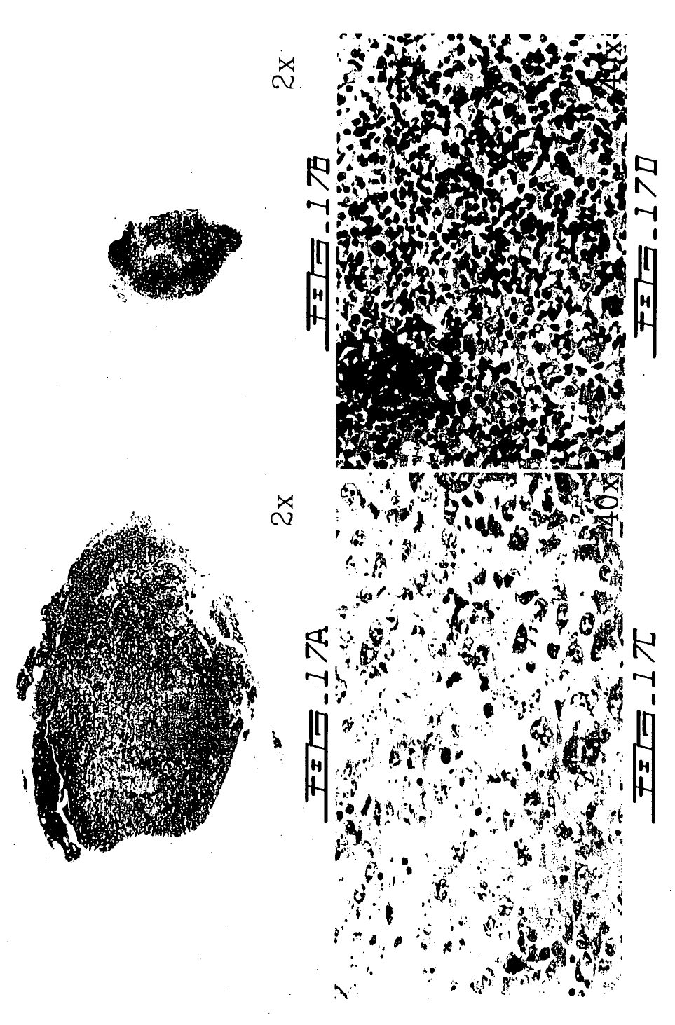

In order to confirm that the fusion protein generated from the novel fusion transgene has immuno-therapeutic uses, one million polyclonal B16-JS4 cells were injected subcutaneously into C57bl/6 mice. As a control, one million B16-AP2 cells were injected in C57bl/6 mice. After 20 days, all mice injected with control B16-AP2 cells had to be sacrificed because the mean tumor volume was more than 800 mm3. In contrast, none of the mice injected with B16-JS4 secreting the fusion protein had a tumor. By day 52 post-implantation, 3 out of 7 mice injected with B16-JS4 cells still did not show any palpable tumor while 4 out of 7 had a mean tumor volume of 25 mm3 (FIG. 16). In FIG. 16, B16 murine melanoma cells were engineered in vitro to express the fusion sequence and GFP (B16-JS4) or to express GFP only (B16-AP2). The level of secretion of the fusion protein was determined in vitro by ELISA on the supernatant of B16-JS4 cells (4 ng of GM-CSF/106 cells/24 h and 2 ng of IL-2/106 cells/24 h). These tumors were then surgically removed at day 52, mounted on paraffin sections and stained with hematoxylin and eosin. The immune infiltration of B16-JS4 tumors was compared to the immune infiltration of B16-AP2 tumors (FIG. 17). Compared to control tumors showing minimal immune infiltration (FIGS. 17A and 17C), tumors secreting the fusion protein were characterized by an intense intratumoral suppurative inflammation (FIGS. 17B and 17D). The inflammation was diffuse through the tumor mass of all JS4 tumors and mainly consisted of neutrophils surrounding degenerated tumor cells.

The immuno-therapeutic effects of the novel synthetic fusion transgene were further compared to those of IL-2 or GM-CSF cDNA. The retrovector plasmid pIL2 (cloned in AP2) or pGMCSF was transfected into GP+E86 packaging cells and the supernatant used to transduced GP+AM12 packaging cells. The supernatant of GP+AM12 was used to transduce B16 murine melanoma cells. Clonal populations of the B16 cells thus generated to produce IL-2 or GM-CSF, as well as clonal populations of B16-JS4 cells secreting the fusion protein, were isolated.

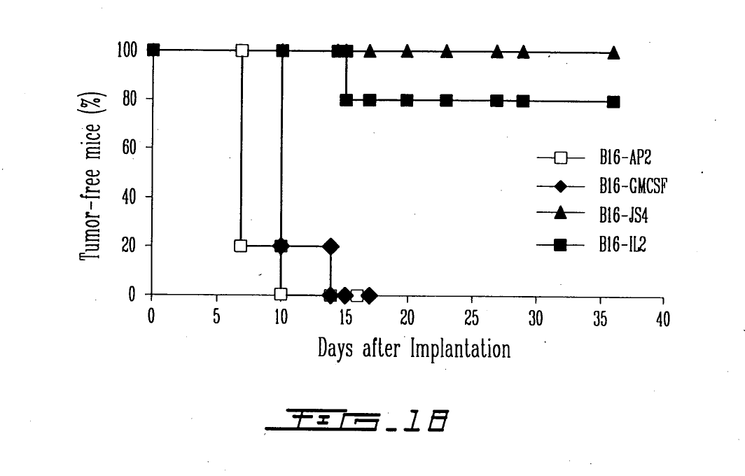

In order to compare the immuno-therapeutic effects of the fusion protein to those of IL-2 or GM-CSF, one million clonal B16 cells secreting IL-2 (B16-IL2), GM-CSF (B16-GMCSF) or equimolar concentration of the fusion protein (B16-JS4) were injected, subcutaneously into C57bl/6 mice. As a control, one million B16-AP2 cells were injected in C57bl/6 mice. At 40 days after injection, all mice injected with B16-JS4 cells secreting the fusion protein were tumor-free, while 20% of mice injected with B16-IL2 and 100% of mice injected with B16-GMCSF had developed a tumor (FIG. 18). In FIG. 18, the level of secretion of the fusion protein was determined in vitro by ELISA on the supernatant of B16-JS4 cells (8ng of GM-CSF/106 cells/24 h and 4 ng of IL-2/106 cells/24 h).

Discussion

In the present application, it is reported the successful engineering of a DNA plasmid encoding for a chimeric protein borne from the fusion of murine GM-CSF and murine IL-2 cDNA. The fusion sequence thereby generated was confirmed by sequence analysis using two distinct DNA primers and revealed the expected presence of a single glycine linker between the 11 amino acid-truncated 3′ end of GM-CSF and the first amino acid of IL-2. When this expression plasmid was transduced into B16 murine melanoma cells, a potent in vivo antitumor effect was observed despite normal cell growth in vitro. 52 days after the s.c. injection of 10⁶ B16-JS4 cells, 3 out of 7 mice failed to develop any tumor. In the 4 mice that did develop cancer, the mean tumor volume was only 25 mm³ after 52 days. Histopathology of the tumors expressing the GMCSF/IL2 fusion sequence revealed an intense intratumoral immune infiltration, mainly consisting of neutrophils and other granulocytes. This suggests that the novel fusion protein is strongly chemotactic for granulocytes, most likely reflecting the GM-CSF subunit activity of the chimera. It is shown herein that the IL-2 portion of the fusion protein is responsible in part, for inhibiting tumor growth. The combined GM-CSF/IL2 have additive beneficial anti-cancer effects such as direct tumoricidal activity and immune recruitment for a “tumor vaccine” effect. It is also shown herein that the humanized version of this murine GMCSF/IL2 fusion DNA sequence will share the same characteristics in humans with cancer. Similarly, species-specific configurations of GMCSF/IL2 fusion gene could be used for veterinary therapeutic purposes.

A second application of this transgene would be as part of a genetic immunoadjuvant of a DNA vaccine for cancer or infectious diseases such as HIV, Hepatitis C or others. Co-expression of an antigen-encoding cDNA and GMCSF/IL2 fusion nucleotide sequence will lead to antigen presentation in a milieu co-generating the GMCSF/IL2 protein, where the GMCSF/IL2 will stimulate a potent immune response (Th1 and Th2) against the presented antigen. Such chimeric cytokine gene could therefore be used as a powerful genetic non-toxic adjuvant to DNA vaccination. Therapeutic use in human clinical applications, as well as agrobusiness applications such as infectious disease of commercially valuable mammals could benefit of such a powerful immunostimulatory cDNA.

It is also proposed that either tumor-targeted delivery of the fusion cDNA (gene) or of the recombinant protein (fusion protein) will have a therapeutic anti-cancer effect in humans. Furthermore, because it has been reported that the highest antibody titers against a DNA vaccine can be obtained when combining the expression of an antigenic peptide to the expression of GM-CSF together with IL-2, the GMCSF/IL2 fusion gene serves as a genetic tool for the generation of polyclonal and monoclonal antibodies as biotechnological reagents. Its use in its current configuration, when co-expressed with a open-reading-frame (ORF) gene, allows the generation of a potent and specific anti-ORF gene product humoral immune reaction. From these immunized animals (mice, rats, goats, etc.) splenocytes could be harvested and utilized to generate novel monoclonal antibody-producing cell lines of commercial interest.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An immuno-therapy conjugate which comprises:
A-c-B
wherein:
A and B are different and are compounds selected from the group consisting of cytokines, chemokines, interferons, their respective receptors or a functional fragment thereof; and
c is a linker consisting of a bond or an amino acid sequence containing from 1 to 100 residues.

2. The conjugate as claimed in claim 1, wherein said cytokine is selected from the group consisting of: GM-CSF, G-CSF, M-CSF, TNF-α, Angiostatin, Endostatin, VEGF, TGF-L, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

3. The conjugate as claimed in claim 1, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

4. The conjugate as claimed in claim 1, wherein said interferon is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

5. An immuno-therapy fusion cDNA encoding the immuno-therapy conjugate of claim 1.

6. The fusion cDNA as claimed in claim 5, wherein said cytokine is selected from the group consisting of: GM-CSF; G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

7. The fusion cDNA as claimed in claim 5, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

8. The fusion cDNA as claimed in claim 5, wherein said interferon is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

9. A vaccine adjuvant for DNA vaccination which comprises the conjugate of claim 1.

10. The vaccine adjuvant as claimed in claim 9, wherein said vaccination is against an infectious organism.

11. The vaccine adjuvant as claimed in claim 10, wherein said infectious organism is selected from the group consisting of: viruses, bacteries, mycobacteria, protozoa and prions.

12. The vaccine adjuvant as claimed in claim 11, wherein said virus is selected from the group of Influenza virus, Hepatitis A virus, Hepatitis B virus, Hepatitis C virus, HIV, Yellow fever virus, Aphthovirus and Filovirus.

13. The vaccine adjuvant as claimed in claim 9, wherein said vaccination is against malignancies, wherein said malignancies having at least one immunogen associated thereto.

14. A vaccine adjuvant for vaccination, which comprises the fusion cDNA of claim 5.

15. The vaccine adjuvant as claimed in claim 14, wherein said vaccination is against an infectious organism.

16. The vaccine adjuvant as claimed in claim 15, wherein said infectious organism is selected from the group consisting of: viruses, bacteria, mycobacteria, protozoa and prions.

17. The vaccine adjuvant as claimed in claim 16, wherein said virus is selected from the group consisting of: Influenza virus, Hepatitis A virus, Hepatitis B virus, Hepatitis C virus, HIV, Yellow fever virus, Aphthovirus and Filovirus.

18. The vaccine adjuvant as claimed in claim 14, wherein said vaccination is against malignancies, wherein said malignancies having at least one immunogen associated thereto.

19. A method for reducing tumor growth in a patient, said method comprising administering to said patient a therapeutically effective amount of the conjugate of claim 1.

20. The method as claimed in claim 19, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

21. The method as claimed in claim 19, wherein said chemokin is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

22. The method as claimed in claim 19, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

23. A method for reducing tumor growth in a patient, said method comprising administering to said patient a therapeutically effective amount of the fusion cDNA of claim 5 using a gene delivery technique.

24. The method as claimed in claim 23, wherein said gene delivery technique is selected from the group consisting of: recombinant viral based vectors and plasmid DNA delivery methods.

25. The method as claimed in claim 23, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

26. The method as claimed in claim 23, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

27. The method as claimed in claim 23, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

28. A method for reducing tumor growth in a patient, said method comprising administering to said patient a therapeutically effective amount of normal autologous patient-derived cells engineered ex vivo to integrate and express the fusion cDNA of claim 5.

29. The method as claimed in claim 28, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

30. The method as claimed in claim 28, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

31. The method as claimed in claim 28, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

32. A method for inhibiting a viral infection in a patient, said method comprising administering to said patient a therapeutically effective amount of the conjugate of claim 1.

33. The method of claim 32, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

34. The method as claimed in claim 32, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

35. The method as claimed in claim 32, wherein said interferons is selected from the group consisting of IFN-α, IFN-β, IFN-γ, IRF-1, IRF-2, IRF-3, IRF-4, IRF-5, IRF-6, IRF-7, IRF-8 and IRF-9 or a functional fragment thereof.

36. The method as claimed in claim 32, wherein said virus is selected from the group consisting of: Influenza virus, Hepatitis A virus, Hepatitis B virus, Hepatitis C virus, HIV, Yellow fever virus, Aphthovirus and Filovirus.

37. A method to inhibit a viral infection in a patient, said method comprising administering to said patient a therapeutically effective amount of the fusion cDNA of claim 6 using a gene delivery technique.

38. The method as claimed in claim 37, wherein said gene delivery technique is selected from the group consisting of: recombinant viral based vectors and plasmid DNA delivery methods.

39. The, method as claimed in claim 37, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

40. The method as claimed in claim 37, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

41. The method as claimed in claim 37, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

42. The method as claimed in claim 37, wherein said virus is selected from the group consisting of: Influenza virus, Hepatitis A virus, Hepatitis B virus, Hepatitis C virus, HIV, Yellow fever virus, Aphthovirus and Filovirus.

43. A method to inhibit a viral infection in a patient, said method comprising administering to said patient a therapeutically effective amount of normal autologous patient-derived cells engineered ex vivo to integrate and express the fusion cDNA of claim 5.

44. The method as claimed in claim 43, wherein said cytokine is selected from the group consisting of: GM-CSF, GM-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

45. The method as claimed in claim 43, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

46. The method as claimed in claim 43, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

47. The method as claimed in claim 43, wherein said viral infection is selected from the group consisting of: Influenza virus, Hepatitis A virus, Hepatitis B virus, Hepatitis C virus, HIV, Yellow fever virus, Aphthovirus and Filovirus.

48. A method to allow production of antigen-specific antibodies, said method comprising the administration of the species-specific fusion cDNA of claim 5 with the cDNA of the said antigen or functional fragment thereof in mammals.

49. The method as claimed in claim 48, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

50. The method as claimed in claim 48, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

51. The method as claimed in claim 48, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

52. A method to improve immune response in a patient, said method comprising administering to said patient a therapeutically effective amount of the conjugate of claim 1.

53. The method as claimed in claim 52, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL8, IL-9, IL-10, IL-11, IL-12, IL-13, IL-14, IL-15, IL-16, IL-17 and IL-18, or a functional fragment thereof.

54. The method as claimed in claim 52, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

55. The method as claimed in claim 53, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

56. Use of a therapeutically effective amount of the conjugate of claim 1 for reducing tumor growth in a patient.

57. The use as claimed in claim 56, wherein said cytokine is selected from the group consisting of: GM-CSF, G-CSF, M-CSF, TNF-α, Angiostatin, Endostatin, VEGF, TGF-(3, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

58. The use as claimed in claim 56, wherein said chemokin is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

59. The use as claimed in claim 56, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

60. Use of a therapeutically effective amount of the fusion cDNA of claim 5 with a gene delivery technique for reducing tumor growth in a patient.

61. The use as claimed in claim 60, wherein said gene delivery technique is selected from the group consisting of recombinant viral based vectors and plasmid DNA delivery methods.

62. The use as claimed in claim 60, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-S, IL-6, IL-7, IL-8, IL-9, IL-10, IL-11, IL-12, IL-13, IL-14, IL-15, IL-16, IL-17 and IL-18, or a functional fragment thereof.

63. The use as claimed in claim 60, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

64. The use as claimed in claim 60, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

65. Use of a therapeutically effective amount of normal autologous patient-derived cells engineered ex vivo to integrate and express the fusion cDNA of claim 5 for reducing tumor growth in a patient.

66. The use as claimed in claim 65, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

67. The use as claimed in claim 65, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

68. The use as claimed in claim 65, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

69. Use of a therapeutically effective amount of the conjugate of claim 1 for inhibiting a viral infection in a patient.

70. The use as claimed in claim 69, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

71. The use as claimed in claim 69, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XQCL, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

72. The use as claimed in claim 69, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

73. The use as claimed in claim 69, wherein said virus is selected from the group consisting of: Influenza virus, Hepatitis A virus, Hepatitis B virus, Hepatitis C virus, HIV, Yellow fever virus, Aphthovirus and Filovirus.

74. Use of a therapeutically effective amount of the fusion cDNA of claim 6 with a gene delivery technique to inhibit a viral infection in a patient.

75. The use as claimed in claim 74, wherein said gene delivery technique is selected from the group consisting of: recombinant viral based vectors and plasmid DNA delivery methods.

76. The use as claimed in claim 75, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

77. The use as claimed in claim 76, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

78. The use as claimed in claim 76, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

79. The use as claimed in claim 76, wherein said virus is selected from the group consisting of: Influenza virus, Hepatitis A virus, Hepatitis B virus, Hepatitis C virus, HIV, Yellow fever virus, Aphthovirus and Filovirus.

80. Use of a therapeutically effective amount of normal autologous patient-derived cells engineered ex vivo to integrate and express the fusion cDNA of claim 5 to inhibit a viral infection in a patient.

81. The use as claimed in claim 80, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

82. The use as claimed in claim 80, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

83. The use as claimed in claim 80, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.

84. The use as claimed in claim 80, wherein said viral infection is selected from the group consisting of: Influenza virus, Hepatitis A virus, Hepatitis B virus, Hepatitis C virus, HIV, Yellow fever virus, Aphthovirus and Filovirus.

85. Use of species-specific fusion cDNA of claim 5 with the cDNA of antigen or functional fragment thereof to allow production of antigen-specific antibodies in mammals.

86. The use as claimed in claim 85, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18, or a functional fragment thereof.

87. The use as claimed in claim 85; wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

88. The use as claimed in claim 85, wherein said interferons is selected from the group consisting of: IFNα, IFN-β, IFN-γ, IRF-1, IRF-2, IRF-3, IRF-4, IRF-5, IRF-6, IRF-7, IRF-8 and IRF-9 or a functional fragment thereof.

89. Use of a therapeutically effective amount of the conjugate of claim 1 to improve immune response in a patient.

90. The use as claimed in claim 89, wherein said cytokine is selected from the group consisting of: GM-CSF, G-CSF, M-CSF, TNF-α, Angiostatin, Endostatin, VEGF, TGF-β, IL-1, IL-2, IL-3, IL-4, IL-S, IL-6, IL-7, IL-8, IL-9, IL-10, IL-11, IL-12, IL-13, IL-14, IL-15, IL-16, IL-17 and IL-18, or a functional fragment thereof.

91. The use as claimed in claim 89, wherein said chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, XCL1, XCL2, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26 and CCL27, or a functional fragment thereof.

92. The use as claimed in claim 89, wherein said interferons is selected from the group consisting of: IFN-α, IFN-β, IFN-γ, IRF-1, IRF-2, IRF-3, IRF-4, IRF-5, IRF-6, IRF-7, IRF-8 and IRF-9 or a functional fragment thereof.


SEQUENCE LISTING (6 sequences)

SEQ ID NO: 1 - DNA, 20 bp, Artificial Sequence
  sequencing primer
acagccagctactaccagac

SEQ ID NO: 2 - DNA, 20 bp, Artificial Sequence
  sequencing primer
cgctaccggactcagatctc

SEQ ID NO: 3 - DNA, 824 bp, Artificial Sequence
  sequence analysis obtained from P1
ttttgatcgcctctngantccnccacggactgtgaaacacaagtcacctatgcggatttcatagacagccttaaaacctttctgactgatggcatgtacagcatgcagctcgcatcctgtgtcacattgacactttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataactatgtacctcctgcttacaacacataaggctctctatttatttggccgcgactctagatcataatcagccataccacatttgtagaggttttacttgctttaaaaaacctcccacacctncccctgaacctgaaacataaaatgaatgcaattgtggtgtaacttgttattgagcttatatggtcaaataagcatacatacaatttacaaataagcatt

SEQ ID NO: 4 - DNA, 869 bp, Artificial Sequence
  sequence analysis obtained from P2
tntttgtcnnnaaccgtttgaaccntcccgatgnnaaattnacttttcctgggcattgtggtctacagcctctcagcacccacccgctcacccatcactntcacccggccttggaagcatgtagaggccatcaaagaanccctgaacctcctggatgacatgcctgtcacattgaatgaagaggtagaagtcgtctctaacgagttctccttcaagaagctaacatgtgtgcagacccgcctgaagatattcgagcagggtctacggggcaacttcaccaaactcaagggcgccttgaacatgacagccagctactaccagacatactgccccccaactccggaaacggactgtgaaacacaagttaccacctatgcggatttcatagacagccttaaaacctttctgactgatggcatgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggatgttctggatttgactaaagcaaaagctttcaattggaagatgctggaatttatcagcaatatagagtactgtgtaaactaanggcttgcacacatttgagtgcaatcatgatngtagcactgtgngacttctgagaatgaagctctg

SEQ ID NO: 5 - protein, 153 aa, Artificial Sequence
  Mouse GM-CSF
SATGLRSRAQASMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPSQK

SEQ ID NO: 6 - protein, 173 aa, Artificial Sequence
  Mouse Interleukin-2
GMYSMQLASCVTLTLVLLVNSAPTSSSTSSSTAEAQQQQQQQQQQQQHLEQLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ